(19) United States
(12) Patent Application Publication
(10) Pub. No.: US 2012/0088846 A1
(43) Pub. Date: Apr. 12, 2012

(54) RADIOPAQUE SHAPE-MEMORY POLYMERS

(75) Inventors: Matthias Lergenmueller, Schluechtern (DE); Sabine Schoen, Herten (DE); Adolf Pfaff, Waldkirch (DE); Karl Friedrich Reichenbach, Waldkirch (DE)

(73) Assignees: ADOLF PFAFF & KARL-FRIEDRICH REICHENBACH GBR, WALDKIRCH (DE); MERCK PATENT GESELLSCHAFT MIT BESCHRANKTER HAFTUNG, DARMSTADT (DE)

(21) Appl. No.: 13/378,056

(22) PCT Filed: May 19, 2010
(86) PCT No.: PCT/EP10/03058

§ 371 Date: Dec. 14, 2011

(30) Foreign Application Priority Data
Jun. 15, 2009 (DE) 10 2009 025 293.2

(51) Int. Cl. A61K 47/32; A61L 31/04; B29C 45/00; A61K 6/08; A61K 47/34; A61L 24/04; A61L 29/04

(52) U.S. Cl. 514/772.4; 523/117; 523/115; 523/113; 514/772.3; 264/328.17

(57) ABSTRACT

The present invention relates to shape-memory polymers which are distinguished by the fact that they comprise BiOCl pigments as X-ray contrast agents. Polymers doped in this way are used, in particular, in medical technology products, such as, for example, stiffening pins for the spinal column, tooth root cones, as bone cement and in catheter materials.

The present invention relates to shape-memory polymers which are distinguished by the fact that they comprise BiOCl pigments as X-ray contrast agents. Polymers doped in this way are used, in particular, in medical technology products, such as, for example, stiffening pins for the spinal column, tooth root-canal cones, as bone cement and in catheter materials.

Radiopaque additives, such as, for example, barium sulfate, zirconium dioxide, zinc oxide and iodine-containing compounds, are employed in a number of medical technology applications in order to render the medical technology product visible by X-ray photograph after use or also to be able to follow it dynamically. Standard possible uses are, inter alia,
barium sulfate in catheter materials
barium sulfate or zirconium dioxide in bone cements
barium sulfate in partly elastic stiffening pins for stabilisation of the spinal column
barium sulfate and/or zinc oxide in gutta-percha cones for tooth root-canal treatment.

For example, radiopacity is one of the numerous requirements of tooth root-canal filling materials. The radiopacity of a root-canal sealer is intended to simplify assessment of the homogeneity of the root-canal filling and the recognition of bubbles and cracks in the root-canal filling.

New X-ray equipment works with increasingly higher energies (kVp) during X-ray irradiation, which, in order to produce the same visibility, either require higher use concentrations of the known filling materials, such as, for example, barium sulfate, or require filling materials which have higher radiopacity.

Since the properties of the material are greatly and in some cases adversely impaired, for example with respect to the elasticity of the material, with increasing use concentration, for example of barium sulfate, materials are being sought which have a more neutral behaviour in this respect and do not influence the material properties or only do so to an insignificant extent.

Shape-memory polymers (SMPs), in particular shape-memory plastics, are materials which are able to change their outer shape under the action of an external stimulus. In medical technology, thermosensitive shape-memory plastics are of particular importance. The shape-memory effect here is not a specific material property of individual polymers; instead, it results directly from the combination of polymer structure and polymer morphology.

Shape-memory plastics are capable of re-adopting their original shape after interim deformation. This memory capacity can be stimulated by an external stimulus, for example by an increase in the ambient temperature or by the incorporation of finely divided magnetic iron-oxide nanoparticles into the plastic, which convert the energy of a magnetic field into heat. For example, the shape-memory polymer, for example a tooth root-canal cone produced therefrom, reaches the so-called switching temperature at 37° C. within a short time after insertion into the human body. The resilience of the polymer then causes the tooth root-canal cone to enlarge to a precisely definable extent, so that the tooth root canal is filled completely and an optimum fit is achieved and the entire root-canal system is durably hermetically sealed in a biocompatible manner.

On use of tooth root-canal cones based on shape-memory polymers, the standard X-ray contrast agent barium sulfate exhibits an adverse effect such that the shape-memory effect no longer becomes fully effective at the individual switching temperature of the polymer and the brittleness of the plastic increases. Cracks and/or fissures thus increasingly occur during cold forming of the plastic. In bone cements, the contrast agents currently used, such as, for example, barium sulfate, no longer have the desired X-ray visibility and have an adverse effect on the elasticity of the cement. Furthermore, the viscosity adjustment of bone cements becomes more difficult with increasing use of radiopaque fillers. In general, the viscosity increases excessively at relatively high use concentrations, meaning that processing, for example injection through cannulas, is made more difficult.

The object of the present invention is to provide an additive having relatively high photon absorption which has good biocompatibility, is non-toxic and can be incorporated very well into a shape-memory polymer and has no or only a slight influence on the shape-memory effect.

Surprisingly, it has now been found that BiOCl pigments are very suitable as radiopaque additives in shape-memory polymers, since, besides their action as X-ray contrast agent, they are non-toxic, do not have an inherent colour and can be incorporated very well into the polymers. Polymers comprising flake-form BiOCl pigments are distinguished by the fact that the use of BiOCl pigments in shape-memory polymers results in elastic materials which can be cold-formed and furthermore have the shape-memory effect with the same or approximately the same recovery dynamics. This means that, at a certain switching temperature (usually body temperature in the case of medical technology products to be incorporated), a pre-defined shape is re-adopted completely after a stretching/drawing/shaping step.

The present invention thus relates to shape-memory polymers which comprise BiOCl pigments as radiopaque additive.

The present invention furthermore relates to the use of the shape-memory polymers according to the invention as material in medical technology, for example as bone cement or for the production of mouldings, such as, for example, tooth root-canal cones, stiffening pins, for example for the spinal column, vascular implants, for example stents, catheters and in implantation aids.

In vertebra reinforcements, the visibility of the reinforcement is increased significantly by the use of BiOCl pigments without impairment of the elasticity. Comparable observations are made on bone cements and catheters, whose flow properties and elasticity respectively are not adversely affected by the use of BiOCl pigments.

Shape-memory polymers are described in the prior art, for example in DE 198 12 160 C1, U.S. Pat. No. 5,962,004, U.S. Pat. No. 5,716,410, WO 99/42528, U.S. Pat. No. 5,458,935, DE 197 55 872 and A. Lendlein, S. Ketch, “Shape-memory polymers”, Angew. Chem. Int. Ed. 2002, 41, 2034-2057.

Suitable shape-memory polymers preferably consist of thermoplastic polyurethanes (TPUs), furthermore of polyvinyl chloride (PVC), polystyrene (PS), polyester, polyvinyl alcohol, polyvinylsiloxane or polycarbonate, and mixtures, and graft polymers and copolymers of the said materials.

Particular preference is given to shape-memory polymers having a Shore hardness of 50A to 80D, very particularly preferably having a Shore hardness of 55A to 75D. The Shore hardness is a material characteristic value of elastomers and plastics and is defined in the standards DIN 53505 and DIN 7868. For tooth root-canal cones, shape-memory polymers, preferably comprising TPU, having a Shore hardness of 55D to 70D are particularly suitable.

The shape-memory polymers preferably exhibit a recovery temperature of 35 to 50° C.

Suitable as implants and for the production of catheters are, in particular, aliphatic thermoplastic polyurethanes, in particular aliphatic, polycarbonate-based thermoplastic polyurethanes, as are commercially obtained in a wide range of hardnesses and colours, for example from Lubrizol Advanced Materials as Thermedics™ polymer products under the trade names

Carbothane® TPU (aliphatic, polycarbonate-based TPU),

Tecoflex® TPU (aliphatic, polyether-based TPU),

Tecophilic® TPU (aliphatic, polyether-based TPU), Tecoplast® TPU, (aromatic, polyether-based TPU),

Tecothane® TPU (aromatic, polyether-based TPU)

Estane® TPU (aromatic, polyester- and polyether-based TPU). All these polymers are suitable for use as medically pure biomaterials. The Carbothanes have extremely high hydrolytic stability and oxidation stability, which indicates excellent long-term biostability and is therefore used, in particular, as reinforcing pins in spinal columns, as stents and for tooth root-canal cones.

Particularly suitable for tooth root-canal cones are thermoplastics, such as, for example, thermoplastic polyurethanes, polyvinyl chloride (PVC), polystyrene (PS), polyesters, polyvinyl alcohols, polyvinylsiloxanes and mixtures, and graft polymers and copolymers of the said materials. The root-canal cones comprising the shape-memory polymers preferably comprise 5-50% by weight of BiOCl pigments, in particular 10-30% by weight, based on the total weight of the compound.

Shape-memory polymers for the production of catheters preferably consist of PU, PVC, polyester, polypropylene or polyethylene and mixtures, and graft polymers and copolymers of the said materials, as well as materials comprising polytetrafluoroethylene (PTFE). The catheters comprising the shape-memory polymers preferably comprise 5-50% by weight of BiOCl pigments, in particular 10-30% by weight, based on the total weight of the catheter material.

Shape-memory polymers for use of vertebra stiffenings preferably consist of thermoplastic polyurethanes, Carbothane® TPU, Tecoflex® TPU, Tecophilic® TPU, Tecoplast® TPU, Tecothane® TPU, Estane® TPU, polyvinyl chloride (PVC), polystyrene (PS), polyesters, polyvinyl alcohols, polyvinylsiloxanes and mixtures, and graft polymers and copolymers of the said materials. The vertebra stiffenings comprising the shape-memory polymers preferably comprise 5-50% by weight of BiOCl pigments, in particular 15-30% by weight, based on the total weight of the compound.

Furthermore, the BiOCl pigments can also be used in shape-memory polymers for the preparation of bone cements. The proportion of BiOCl pigment in the bone cement (polymer) is preferably 5-50% by weight, in particular 10-30% by weight, based on the total weight of the bone cement.

However, the use concentration of the BiOCl pigment in shape-memory polymers is dependent on the polymer employed. In general, the BiOCl pigments are added to the polymer in amounts of 5-50% by weight, preferably 10-40% by weight, in particular 10-30% by weight, based on the total weight.

Besides the function as X-ray contrast agent, the BiOCl pigment can also serve as filler and thus positively influence the deformability, elasticity, stretchability of the plastic. If the BiOCl pigment is employed merely as X-ray contrast agent, the use concentration are in the range 5-50% by weight, preferably 10-40% by weight and very particularly preferably 15-30% by weight, based on the total weight of the polymer or polymer preparation.

BiOCl pigments are known, for example, from DE Patent 10 03 377, U.S. Pat. No. 2,975,053, DE 24 11 966, EP 0 496 686 B1 and DE 43 05 280 A1 and are commercially available and are offered, for example, by Merck KGaA, Germany, under the trade names Bi-Flair®, Biron®, RonaFlair™ and by BASF under the trade name Meerlite®. The commercially available BiOCl pigments have particle sizes of 1-50 μm. For use of the BiOCl pigments in memory-shape plastics, BiOCl pigments having particle sizes of 2-50 μm, in particular 5-20 μm and very particularly preferably <15 μm, are preferably suitable. Owing to the diverse production possibilities, the flake-form BiOCl pigments are available with different optical properties, from matt to glossy and from transparent to opaque. The size of the individual particles for the highly glossy BiOCl pigments is preferably 6-20 μm, in particular 8-18 μm and very particularly preferably 10-16 μm.

The BiOCl pigments are uncoated, are in the form of flakes and are generally added to the monomer in the form of loose powders in the preparation of shape-memory polymers.

The shape-memory polymers according to the invention are prepared, for example, by compounding the BiOCl pigment into the plastic. The BiOCl pigment can furthermore be added in powder form immediately before or during polymerisation of the selected plastic and mixed, so that separate compounding-in is avoided. The latter process is preferred, since this gentle incorporation means that the flake structure of the BiOCl pigment suffers significantly less damage.

The shape-memory polymer doped in accordance with the invention is generally prepared by initially introducing the plastic granules in a suitable mixer, wetting them with any additives and then adding and mixing in the BiOCl pigment. During incorporation of the BiOCl pigment, adhesives, organic polymer-compatible solvents, stabilisers and/or surfactants which are heat-stable under the working conditions can optionally be added to the plastic granules. The plastic is generally pigmented via a colour concentrate (masterbatch) or compound. The resultant mixture can then be processed directly in an extruder or injection-moulding machine. The mouldings formed on processing exhibit a very homogeneous distribution of the BiOCl pigment.

The invention also relates to moulded parts, in particular for medical technology products, consisting of the shape-memory polymer According to the invention comprising BiOCl pigments.

The shape-memory polymers doped in this way are particularly suitable for the production of tooth root-canal cones, reinforcing pins for the spinal column, catheter materials, vascular implants, for example stents, implantation aids.

In a preferred embodiment, the implants comprising the shape-memory polymer according to the invention comprise at least one medical active compound, such as, for example, cytostatics, antiangiogenic active substances, corticoids, NSAID, heparin, hirudin, which is, if desired, released to the surrounding tissue in high concentration and over an extended period. The active compounds can be added directly to the monomer during polymerisation and are then in homogeneously distributed form in the plastic powder or plastic granules or can be added in the desired amount to the moulding during processing of the polyurethane melt or polyurethane solution. The active compound(s) are preferably dissolved or dispersed in the polymer, it being possible for the dissolution of the active compound to be carried out both in the melt and in the organic solution of the polymer. Thus, it is possible to achieve admixing of up to 30% by weight of active compound in the polyurethane. The processing is carried out as described above by extrusion or injection moulding, where only thermally resistant active compounds can be used in the extrusion or injection-moulding process.

The present invention likewise relates to the use of the radiopaque shape-memory polymers according to the invention as implant material, for example for the production of tooth root-canal cones, stiffening pins, for example for spinal columns and costal bones, hip and knee joints, for the preparation of bone cements, vascular implants, stents, catheters, such as, for example, bladder catheters, vein catheters, central-vein catheters, cardiac catheters, for the production of implantation aids, for the production of reference pins for various applications in the area of medicine.

The following examples are intended to explain the invention in greater detail, but without limiting it. Above and below, percentage data denote per cent by weight. All temperatures are indicated in degrees Celsius.

### EXAMPLES

### Example 1

### Production of Plastic Parts by Injection Moulding

The three shape-memory Carbothane® plastics
PC 3572D (hard)
PC 3595A (soft)
PC 3555D (medium) from Lubrizol are each compounded with 45% of RonaFlair™B-50 (BiOCl pigment having a particle size of 2-35 μm from Merck KGaA) and granulated. The granules are introduced into the hopper of the injection-moulding machine, warmed and injected into the cavities of the mould under high pressure. In this way,
tooth root-canal cones
stents
stiffening pins
reference pins for various applications in the area of medicine are produced.

The final products are distinguished by very good radiopacity.

### Example 2

### Production of Plastic Parts by Injection Moulding

Analogously to Example 1, Carbothane PC 3572D (Lubrizol) is compounded with 40% of RonaFlair™ LF-2000 (BiOCl pigment having a particle size of 2-35 μm from Merck KGaA) and granulated. The granules are introduced into the hopper of the injection-moulding machine, warmed and injected into the cavities of the mould under high pressure. In this way,
tooth root-canal cones
stents
stiffening pins
reference pins for various applications in the area of medicine are produced.

The final products are distinguished by very good radiopacity.

### Example 3

### Production of Plastic Parts by Injection Moulding

Analogously to Example 1, Carbothane PC 3572D (Lubrizol) are compounded with 45% of RonaFlair™ Fines (BiOCl pigment having a particle size of 2-35 μm from Merck KGaA) and granulated. The granules are introduced into the hopper of the injection-moulding machine, warmed and injected into the cavities of the mould under high pressure. In this way,
tooth root-canal cones
stents
stiffening pins
   are produced.

The final products are distinguished by very good radiopacity.

### Example 4

### Production of Catheter Tubes by Extrusion

Carboethane PC 3572D from Lubrizol is admixed with 25% of RonaFlair™ B-50 (BiOCl pigment having a particle size of 2-35 μm from Merck KGaA) and converted into a viscous consistency by warming and subsequently introduced into an extruder. The viscous plastic material with compacted and forced through a shaping aperture into the extrusion mould. The extrusion mould is a hollow mould into which the plastic material is forced on one side through the extruder and which it leaves on the other side as a finished tube. For this purpose, the material flow is split within the mould by a mandrel support and flows around the mandrel, which shapes the cavity in the tube. Whereas the tube volume is determined by the mandrel, the diameter of the die through which the material flow exits is responsible for the external cross section of the tube. The material-specific shrinkage properties of the plastic during cooling influence the dimensions of the end product.

The final product is distinguished by its excellent X-ray opacity.

1. Shape-memory polymers, characterised in that they comprise BiOCl pigments.

2. Shape-memory polymers according to claim 1, characterised in that the BiOCl pigment is in flake form.

3. Shape-memory polymers according to claim 1, characterised in that the BiOCl pigment has particle sizes of 2-50 μm.

4. Shape-memory polymers according to claim 1, characterised in that the BiOCl pigment is employed in powder form.

5. Shape-memory polymers according to claim 1, characterised in that the proportion of BiOCl pigment in the polymer is 5-50% by weight, based on the total weight of the polymer or polymer mixture.

6. Shape-memory polymers according to claim 1, characterised in that the polymer is selected from the group of the thermoplastics.

7. Shape-memory polymers according to claim 1, characterised in that the thermoplastic is selected from the group polyurethane (TPU), polyester, polyvinyl alcohol, polyvinylsiloxane, polycarbonate.

8. Shape-memory polymers according to claim 1, characterised in that they have a Shore hardness of 50A to 80D.

9. Shape-memory polymers according to claim 1, characterised in that they have a recovery temperature of 35 to 50° C.

10. Shape-memory polymers according to claim 1, characterised in that they comprise at least one medical active compound.

11. Process for the preparation of shape-memory polymers according to claim 1, characterised in that the BiOCl pigment is compounded into the plastic or added during polymerisation of the selected plastic, and the resultant mixture is processed in an extruder or injection-moulding machine, optionally with addition of further additives.

12. Process for the preparation of shape-memory polymers according to claim 11, characterised in that at least one medical active compound is additionally added to the plastic powder or compound, and the resultant mixture is processed in an extruder or injection-moulding machine, optionally with addition of further additives.

13. A method comprising producing root-canal cones, stiffening pins, stents, vascular implants, catheter materials, implantation aids, or reference pins for applications in medical technology with shape-memory polymers according to claim 1.

14. Mouldings consisting of a shape-memory polymer according to claim 1.
